(19) United States
(12) Patent Application Publication
(10) Pub. No.: US 2006/0111622 A1
(43) Pub. Date: May 25, 2006

(54) Apparatus and method for monitoring deep tissue temperature using broadband diffuse optical spectroscopy

(76) Inventors: Sean Merritt, Lake Forest, CA (US); Bruce J. Tromberg, Irvine, CA (US); Albert E. Cerussi, Lake Forest, CA (US); Anthony J. Durkin, Irvine, CA (US)

Correspondence Address: MYERS DAWES ANDRAS & SHERMAN, LLP, 19900 MACARTHUR BLVD.,, SUITE 1150, IRVINE, CA, 92612, US

(21) Appl. No.: 11/246,369

(22) Filed: Oct. 7, 2005

Related U.S. Application Data
(60) Provisional application No. 60/617,402, filed on Oct. 7, 2004.

(51) Int. Cl. G06F 19/00; A61B 5/00

(52) U.S. Cl. 600/315; 702/19

(57) ABSTRACT

A method for noninvasively determining deep tissue temperature comprises measuring data relating to spectral shifts of chromophore absorption in tissue using broadband diffuse optical spectroscopy and generating a temperature reading corresponding to the spectral shift of an absorption peak of the chromophore. A bound water correction is made to the spectral shift. A frequency domain measurement at multiple wavelengths is made to determine the absolute absorption and scattering values between 600 and 1050 nm. The measurement of an absolute absorption comprises measuring an absolute absorption coefficient of selected tissue and further comprising deducing concentrations of tissue composition including lipids, deducing information related to heterogeneity and integrity of tissue matrix, and deducing temperature heterogeneity related to vulnerable plaque in vascular tissue. The measurement comprises making a measurement in the range of 600-1100 nm to interrogate a vessel wall in the presence of blood.

### RELATED APPLICATIONS

The present application is related to U.S. Provisional Patent Application Ser. No. 60/617,402, filed on Oct. 7, 2004, which is incorporated herein by reference and to which priority is claimed pursuant to 35 USC 119.

### GOVERNMENT RIGHTS

This invention was made with Government support under Grant No. RR01192, awarded by the National Institutes of Health. The Government has certain rights in this invention.

### BACKGROUND OF THE INVENTION

1. Field of the Invention

The invention relates to the field of optical measurement of tissue parameters using diffuse optical spectroscopy (DOS). In addition the invention relates to a method of measuring temperature in tissue for assessing the health of vasculature and, in particular, identifying regions of vulnerable plaque prior to rupture.

2. Description of the Prior Art

For successful noninvasive or minimally invasive thermal tissue therapies, it is imperative to have a feedback modality for monitoring deep tissue temperature noninvasively. The necessity for tissue temperature monitoring is apparent in both superficial and deep tissue thermal therapies. For example superficial thermal therapies such as skin remodeling apply RF or laser energy to the skin in order to change the collagen structure in the dermal layer. With these treatments the dermal layer is targeted to reach 65-75° C. causing the collagen to denature while cooling is applied to the surface of the skin to maintain the epidermis within a temperature range of 35-45° C.

There are also thermal therapies for deeper tissue structures such as breast cancer treatment. Hyperthermia and coagulative therapies treat the tumor with microwaves or RF to raise the temperature of the cancer cells. For hyperthermia the cancer cells are elevated to a temperature of 40-44° C. and for coagulative therapies the tumor is heated above 60° C. For each of these methods it is critical that the temperature of the normal tissue is kept below 40° C. in order to avoid unnecessary damage.

Other potential applications involve measurement of unperturbed tissue temperature. Breast thermography is used to measure the difference in temperature at the tissue surface between normal and cancerous breast. Needle temperature probes have been used to determine a maximum temperature difference of ˜3° C. between tumor and normal breast tissue. Currently broadband DOS is used to characterize breast tumor tissue through measurements of tissue chromophores and scattering properties, but the addition of tissue temperature will enhance the ability of tissue characterization. Most notably, the contrast that arises between tumor and normal breast tissue temperature is related to increased vascularization and blood flow. This is already characterized through hemoglobin measurements using broadband DOS, but the ability to measure temperature should make vascularity characterization more robust.

The temperature dependent spectral changes of water are well known. As water is heated the thermal motion of the water molecules begins to overcome the force of hydrogen bonds that connect the molecules. As a result these bonds are broken and the subsequent spectral effect include (1) the water absorption peak at ˜976 nm blue shifts, (2) the water peak is narrowed and (3) the water peak increases in intensity. These spectral changes have been well characterized within the near-infrared region and have been measured directly in a spectrophotometer.

Despite potential applications and well characterized changes, there has been only one thorough study that investigated the ability to determine tissue temperature optically. Hollis investigated the use of an optical technique to measure deep tissue temperature for the purpose of monitoring neonatal brain temperature. She used a CW technique and measured reflectance over the spectral range of 650-980 nm. Within this spectral window water absorption peaks are located at ˜740, ˜840 and ˜976 nm. The primary focus was on the absorption peaks at 740 and 840 nm because the lower absorption of these peaks relative to the 976 nm peak allow for deeper penetration of light. Hollis used the technique of principal component regression (PCR) to calibrate the temperature response of water spectra in order to determine parameters associated with the temperature response that could then be used for fitting of tissue temperature. Measurements performed on an adult forearm had a standard error of prediction of 1.2° C. determined for the recovered temperature. Hollis concluded that in order to improve results scattering should be accounted for and that using the water peak at ˜976 nm would provide more contrast although it would limit the light penetration depth in the tissue.

Arteriosclerosis is an inflammatory disease. Inflammatory processes play a role in the initiation of plaque development and the early stages of the disease as well as in complex plaques and complications such as intra-arterial thrombosis. A method to detect inflammation in coronary arteries has the potential to characterize both local and systemic activation of atherosclerotic plaque disease. Experimental work by others has suggested that thermal heterogeneity is present in atherosclerotic plaques and that increased temperature is found at the site of inflammatory cellular-macrophage-infiltration. Increases in thermal heterogeneity appear to be associated with a comparably unfavorable long-term prognosis. Intracoronary thermography has the potential to provide insights into location and extent of inflammation as well as the prognostic consequences.

Many approaches have been proposed to detect vulnerable plaque. MRI, nuclear imaging techniques, endovascular ultrasonography, angiography, angioscopy, infrared spectroscopy, and cardiovascular wall temperature measurements may be used to determine the presence and location of carotid, aortic and coronary atherosclerotic plaques. Included in such devices are thermal sensing catheters, as well as infrared and optical coherence tomography (OCT) catheters

A method for assessing the temperature of deep tissue in-vivo using non-ionizing radiation would have great utility in this medical application. The use of nonionizing radiation, including but not limited to the visible, near-infrared, and infrared spectral regions could offer a significant advance, if a method for noninvasively measuring deep tissue temperatures could be found.

Zuluaga US Patent Application 20050107706 (May 19, 2005) describes an intraluminal spectroscope with wall-contacting probe. The apparatus, which detects vulnerable plaque within a lumen defined by an intraluminal wall, includes a probe through which an optical fiber extends. An coupler in optical communication with the fiber is configured to atraumatically contact the intraluminal wall. A light source provides light to the fiber for illuminating the wall and a detector coupled to the fiber receives light from within the wall. Zuluaga relies on infrared wavelengths in the range 1100-2400 nm and proposes to characterize plaque based on spectroscopic information. This method relies on empirical calibration based models such as PLS to quantify composition. No claims is made to be able to determine temperature and there is no discussion of shifts in spectral features.

Kokate US Patent Application 20030233052 (Dec. 18, 2003) describes a catheter with thermal sensor for detection of vulnerable plaque. An elongate medical device which includes an elongate shaft to which a substrate is fixed proximate the distal end, and a plurality of sensors are disposed on the substrate. Each sensor is coupled to a switching device. The switching devices are disposed on the substrate. Kokate relies on contact between the catheter delivered sensor and the vessel wall. In this case the sensor is not optical and does not rely on spectral shift.

Madjid et. al., “Thermal detection of vulnerable plaque.” Am J Cardiol. 2002 Nov. 21;90(10C):36L-39L (Division of Cardiology/Internal Medicine, University of Texas-Houston Health Science Center, USA) shows that inflamed atherosclerotic plaques give off more heat and that vulnerable plaques may be detected by measuring their temperature. Plaque temperature is correlated directly with inflammatory cell density and inversely with the distance of the cell clusters from the luminal surface. It is inversely related to the density of the smooth muscle cells. No significant association between temperature heterogeneity and presence of Chlamydia pneumoniae in plaque or the gross color of human atherosclerotic carotid plaques was found. ph heterogeneity in plaques from human carotid artery and aortas of Watanabe atherosclerotic rabbits and apolipoprotein E-deficient mice was found. Areas with lower pH had higher temperature, and areas with a large lipid core showed lower pH with higher temperature, whereas calcified regions had lower temperature and higher pH. A thermography basket catheter was used to show in vivo temperature heterogeneity in atherosclerotic lesions of atherosclerotic dogs and Watanabe rabbits. Thermal heterogeneity was later documented in human atherosclerotic coronary arteries. Temperature difference between atherosclerotic plaque and healthy vessel wall is related to clinical instability. It is correlated with systemic markers of inflammation and is a strong predictor of adverse cardiac events after percutaneous interventions. Thermography is the first in a series of “functional” imaging methods and is to be used in clinical trials. It may be useful for a variety of clinical and research purposes, such as detection of vulnerable plaques and risk stratification of vulnerable patients.

While Madjid discusses the relationship between temperature and vessel wall heterogeneity at length and recognizes a correlation between vessel pH and temperature is recognized, there is no discussion of using spectroscopic means to deduce temperature.

Diamantopoulos, “Arterial wall thermography”, J Interv Cardiol. 2003 June;16(3):261-6 (Cardiology Department, University Hospital Gasthuisberg, Leuven, Belgium.) describes that atherosclerotic plaque is considered vulnerable when it is at higher risk of inducing acute cardiac events. The early detection and follow-up of the vulnerable plaque are crucial to prevent these events from happening. As of 2003 there are no proven techniques to detect such a plaque. Arterial wall thermography, tracing the heat signature of the activated macrophages was thought to be a promising method for this purpose. However, the difficulties of applying such a method in vivo should not be neglected. Prior technologies propose several potential thermographic methods. They can be generally categorized as noninvasive and invasive. Magnetic resonance thermometry (MRT) was considered the most important noninvasive method. It is accurate, and reproducible, but is unfortunately hampered by resolution limitations due to the size and motion of the target vessels. The “infrared” and the “contact-sensor” were thought to be the most important invasive thermographic methods. Mainly due to the difficulties of infrared radiation in penetrating the flowing blood, the contact thermographic methods were then seen to be the most feasible. The superiority of thermal mapping of the arterial wall versus the localized temperature measurements was clear. The use of multiple thermal sensors arranged around the vessel's circumference and the application of motorized catheter pullback, not only ensure a large area of coverage, but also enable thermal maps and vascular thermoanatomical reconstructions to be built by using computer technology. It was then expected that arterial thermography will undoubtedly initiate debate, mainly concerning the most appropriate therapy for the vulnerable plaque.

Several potential thermographic methods for investigation vulnerable plaque are discussed by Diamantopoulos. There is mention of using infrared techniques to probe vessel wall. However, the wavelength range of the infrared discussed in this review is in the 3-5 micron range and the 10 micron range. All of these infrared wavelengths are relevant to very superficial interrogation of the vessel wall. None of these wavelengths are capable of penetrating any significant thickness of blood and still returning a meaningful signal. Contact between the probe and vessel wall is a likely condition for these methods to work. There is no discussion of using spectral shift in absorption as an indicator of temperature.

Khan et. al. “Tissue pH determination for the detection of metabolically active, inflamed vulnerable plaques using near-infrared spectroscopy: an in-vitro feasibility study.” Cardiology. 2005;103(1):10-6. Epub 2004 Nov. 2 (Department of Surgery, University of Massachusetts Medical School, Worcester, Mass., USA) describe the detection of vulnerable plaques as the underlying cause of myocardial infarction is at the center of attention in cardiology. It was known then that infiltration of inflammatory cells in atherosclerotic plaques renders these plaques relatively hot and acidic, with substantial plaque temperature and pH variation. The objective was then to determine whether near-infrared diffuse reflectance spectroscopy (NIRS) could be used to non-destructively measure the tissue pH in atherosclerotic plaques. NIRS and tissue pH electrode measurements were taken on freshly excised carotid plaques maintained under physiological conditions. The coefficient of determination between NIRS and the pH microelectrode measurement was 0.75 using 17 different areas. The estimated accuracy of the NIRS measurement was 0.09 pH units. These results demonstrate the feasibility of using NIRS tissue pH in freshly excised atherosclerotic plaques in light of marked pH heterogeneity and motivated future in-vivo investigations on pH measurement of atherosclerotic plaques.

The Khan instrument does not model the propagation of light through the medium and only measures the raw reflectance. Their invention therefore relies on the PLSR and SMLR analysis techniques relate reflectance to pH of the vessel wall and then correlates this to temperature. These analysis techniques use previous measurements to determine a calibration set and it works if the current measurement is similar to measurements within the calibration set, but the technique breaks down when a measurement is sufficiently different from the data in the calibration set. There is no mention in this work of using spectral shift in absorption coefficient an indicator of temperature.

What is needed is a method of monitoring deep tissue temperature and a method for detecting vulnerable vascular plaque.

### BRIEF SUMMARY OF THE INVENTION

The illustrated embodiment of the invention is an apparatus and a method of monitoring deep tissue temperature using broadband diffuse optical spectroscopy (DOS). In the illustrated embodiment deep tissue monitoring contemplates noninvasive monitoring of the order of 1-4 cm in depth into the tissue, but may include shallower or even deeper ranges depending on the wavelength and intensity of light used, and the nature of the tissue. It separates scattering from absorption and provides an absolute measure of the tissue absorption from 650-1000 nm. In one embodiment the method focuses on the ˜976 nm water absorption peak for greater contrast in tissue temperature. Measurements on tissue phantoms and an animal model are presented for validation of the technique.

The invention can also be used to identify chromophores and quantify their concentrations in deep tissue.

In another aspect of the invention an algorithm is employed whereby the chromophores and their temperatures are quantified. The absolute absorption spectrum is measured according to the invention. A chromophore composition and temperature is postulated for the tissue as a first guess. Taking the absorption spectrum from a library, the spectra for the postulated composition and temperature is subtracted from the measured spectrum, leaving in theory the spectrum for water. According to another aspect of the invention this spectrum is corrected for bound water. Based on a stored spectrum for the temperature shift for unbound water, a new temperature is then interpolated from the stored spectrum. Using the new calculated temperature, the spectrum for the postulated composition is again calculated and then subtracted from the measured spectrum corrected for bound water. This in turn yields a new calculated temperature deduced from the interpolated stored library temperature shifted spectra for the postulated composition. The iteration continues until the temperature converges to within a predetermined error range, which in the illustrate embodiment has been chosen at 0.1° C., but could also be set at other levels of precision.

The spectra above are chosen from interpolated spectra in a stored library of spectra using a least squares fit as described in Tromberg et al, U.S. Patent Application 20030023172. In the case where the postulated composition is in error, this same spectra matching process can be used to search a library of other chromophores until one is found which provides a best fit with the measure data. In this manner unknown chromophores can be identified. The same process is also capable of quantifying the concentration of the chromophore.

Still further according to yet another aspect of the invention, temperature differences in tissue may be mapped. As described below vulnerable plaque in the vascular system can be mapped in a quantified manner for improved diagnostic methods using the invention.

According to another aspect of the invention, tissue may be artificially heated and its temperature dependent shift of the absolute optical absorption measured, again as a means of identifying and quantifying tissue components.

The illustrated embodiment includes an apparatus and method for determining deep tissue temperature non-invasively using broadband diffuse optical spectroscopy (DOS). The temperature dependent water absorption spectrum of pure water is measured. As water temperature is increased the water absorption peak at ˜980 nm is blue shifted, narrows and increases in intensity. These measured changes were incorporated into an algorithm that fits a tissue absorption spectrum for temperature. A bound water correction was implemented using an empirical correction.

From the tissue data acquired with broadband DOS an empirical bound water correction was developed to account for the shifting and broadening of the water spectra. Bound water are those molecules that are bound by hydrogen bonds to other molecules, such as other water molecules or hydrated to proteins. It was found that above 935 nm the tissue water absorption spectrum was linearly shifted to higher wavelengths relative to the pure water spectrum. The correction provides an improved chromophore fit overall, especially for the water and lipid chromophores which are determined within the wavelength range at which the bound water effects are occurring. Since the bound water effects are not observed below 935 nm, this wavelength is used as a pivot point at which the correction begins. The wavelengths above 935 nm are shifted using the following linear equation:

$$shifted WL = [ \frac{wpwl + bws - 935 nm}{wpwl - 935 nm} ] \times WL - 935 nm [ \frac{bws}{wpwl - 935 nm} ]$$

Where wpwl is the peak wavelength of the free water spectrum, bws is the bound water shift defined as the peak-to-peak distance between the shifted spectra, WL are the wavelengths above 935 nm before the shift correction. A positive bws occurs when the tissue water is red-shifted from the pure water spectrum. When this is the case the shifted WL is less than the normal WL, which causes the corrected pure water spectrum to be broadened in a similar manner to what is observed in tissue water. The bws used in the correction was 8.9 nm and it is clear that the corrected water spectrum matches the tissue water spectrum more closely than does the uncorrected pure water.

Broadband DOS measurements were acquired on a liquid phantom and an animal model. The phantoms were elevated from room temperature up to 60° C. and the animal model was decreased from body temperature to ˜24° C. Following the correction of systematic errors in the recovered broadband DOS temperature the average error in the recovered temperature was determined to be 0.7±0.6° C.

Broadband DOS uses a frequency domain technique at multiple laser diode wavelengths to determine the absolute absorption and scattering values at these wavelengths. A broadband measurement is then incorporated by scaling with the frequency domain measurements in order to provide an absolute quantitative absorption spectrum in the wavelength region between 600 and 1050 nm. The prior art above utilized optical techniques such as spatially-resolved optical measurements, which provide relative measured spectra within the near-infrared wavelength region, but does not include an absolute measurement of tissue absorption. An absolute measurement of tissue absorption is needed in order to properly separate temperature related changes form scattering and concentration changes that will also take place within the tissue.

Explicit advantages of the illustrated embodiment of the inventive method include the fact that the method uses models to describe the propagation of light through the medium and from this we determine the absolute scattering and absorption information of the medium.

From absorption coefficient, we can deduce concentrations of vessel contents including lipids (as in lipid pool underneath fibrous cap). From scattering coefficient, we can deduce information related to the heterogeneity and integrity of the vascular matrix. From spectral shifts of the spectral band related to water we can deduce temperature heterogeneity related to vulnerable plaque. By working in the 600-1100 nm spectral window, we have the capability of interrogating vessel wall in the presence of blood (something that is not possible in the 1100-2400 nm range because attenuation due to water will extinguish the signal).

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIGS. 1a and 1b are graphs comparing the adjustment of the spectral window used in the temperature algorithm. FIG. 1a is a plot of correlation coefficient between thermistor temperature and broadband DOS temperature. FIG. 1b is plot of the average temperature difference (▪) and the standard deviation of the temperature differences (∘). The upper bound wavelength is fixed at 1000 nm while the lower bound wavelength is varied from 975 to 935 nm.

FIGS. 2a and 2b are plots comparing the adjustment of the spectral window used in the temperature algorithm. FIG. 2a is a plot of correlation coefficient between thermistor temperature and broadband DOS temperature. FIG. 2b is plot of the average temperature difference (▪) and the standard deviation of the temperature differences (∘). The lower bound wavelength is fixed at 945 nm while the lower bound wavelength is varied from 975 to 1015 nm.

FIG. 3 is a plot of thermistor (▪) and broadband DOS (∘) temperature measurements on a liquid phantom. The error bars for the thermistor measurements were smaller than the symbols for the error bars.

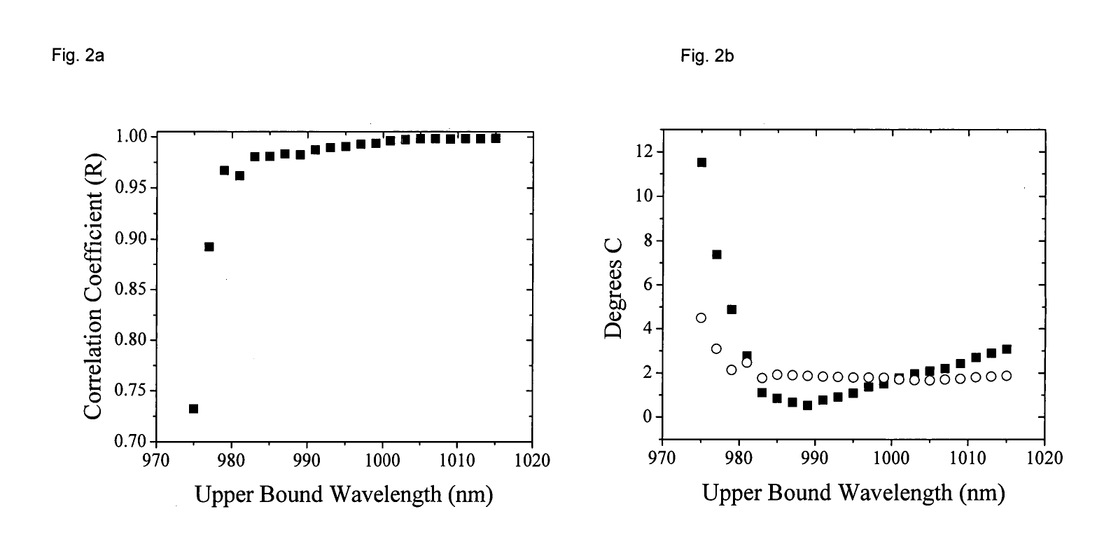

FIG. 2 is a plot of temperature difference between thermistor and broadband DOS for the liquid phantom in Fig (▪) and two separate liquid phantoms measured on separate days (∘) and (Δ).

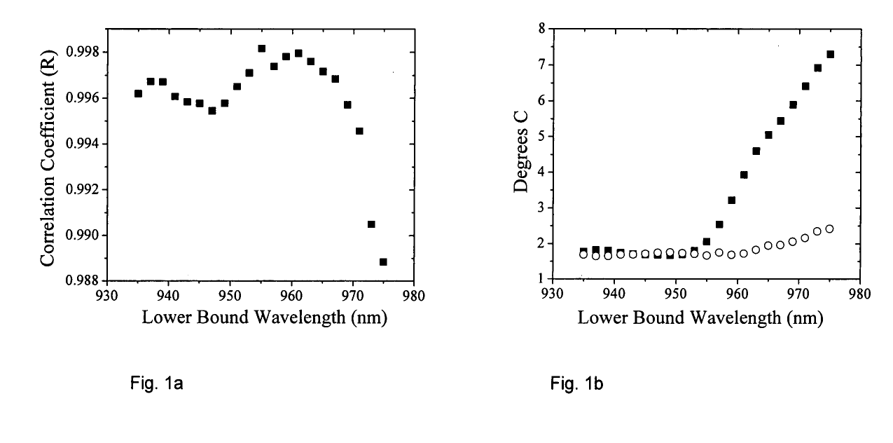

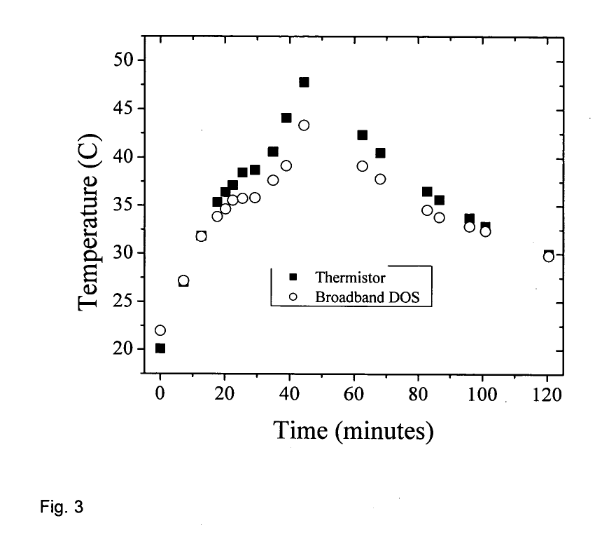

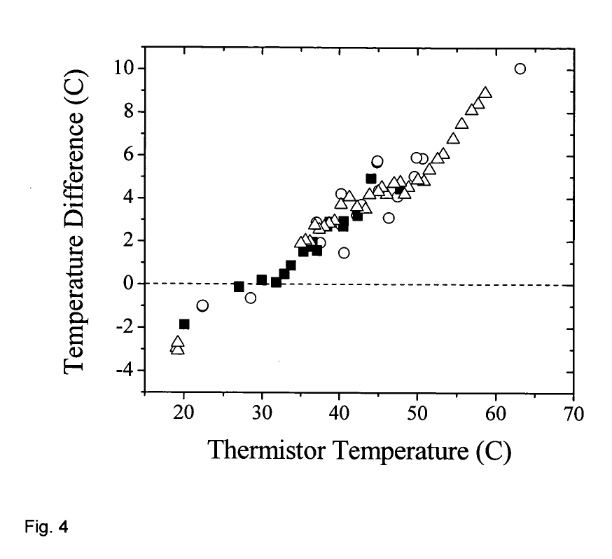

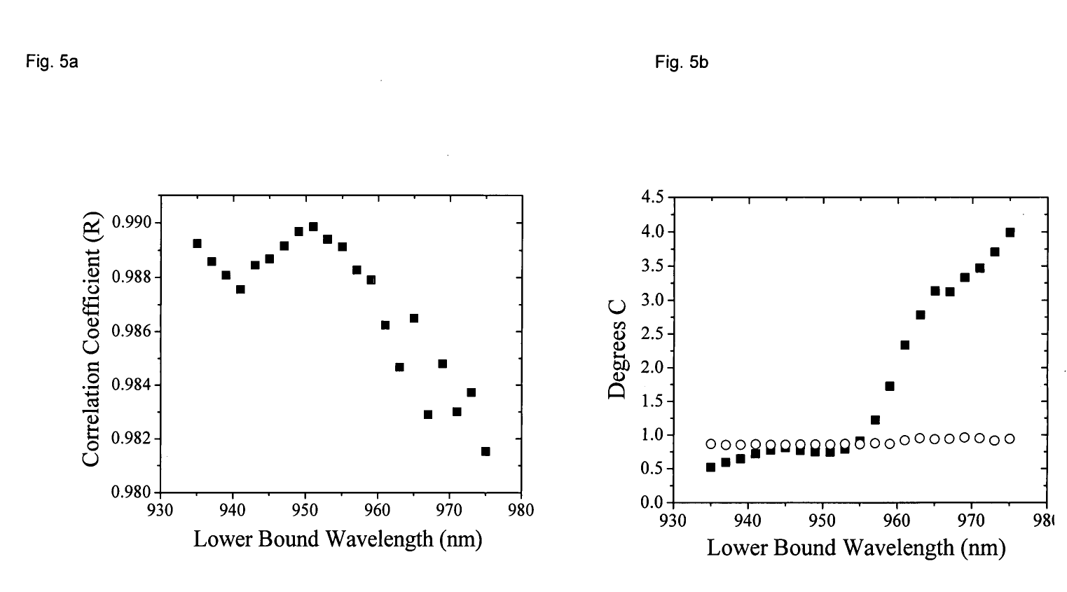

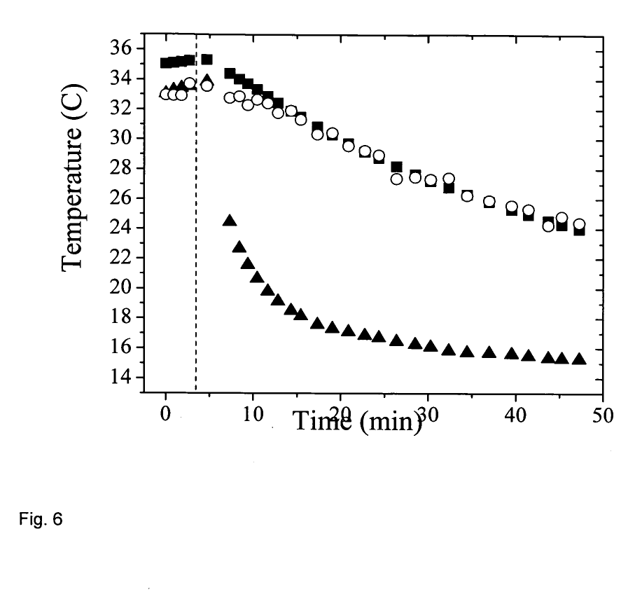

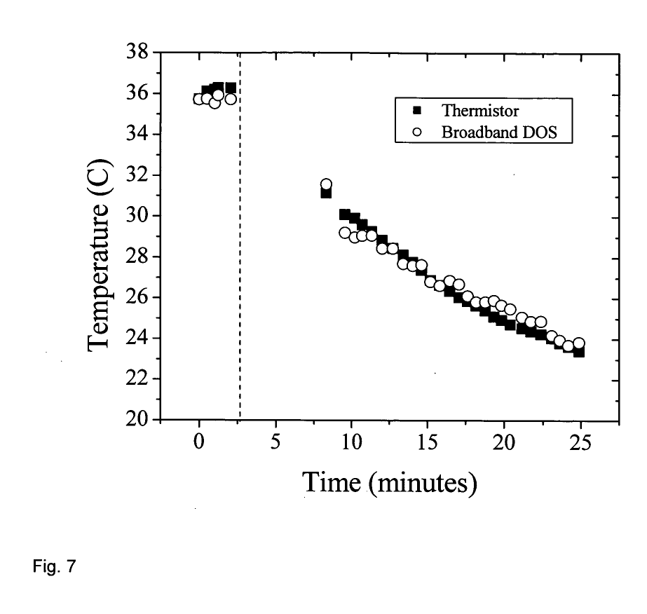

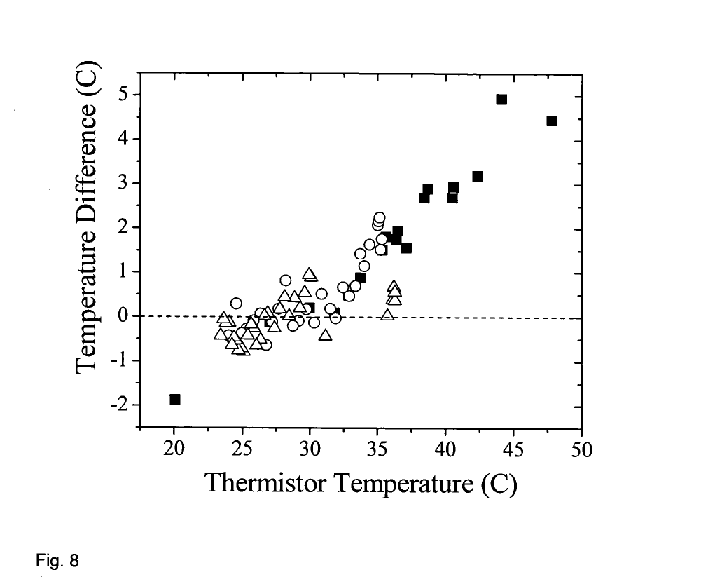

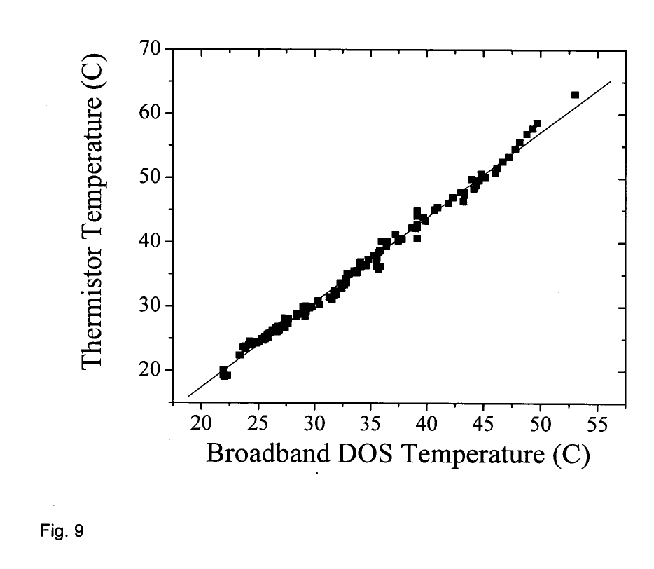

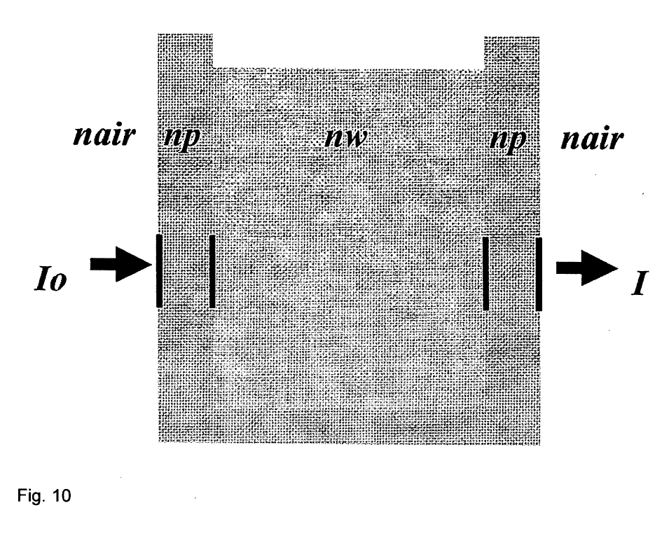

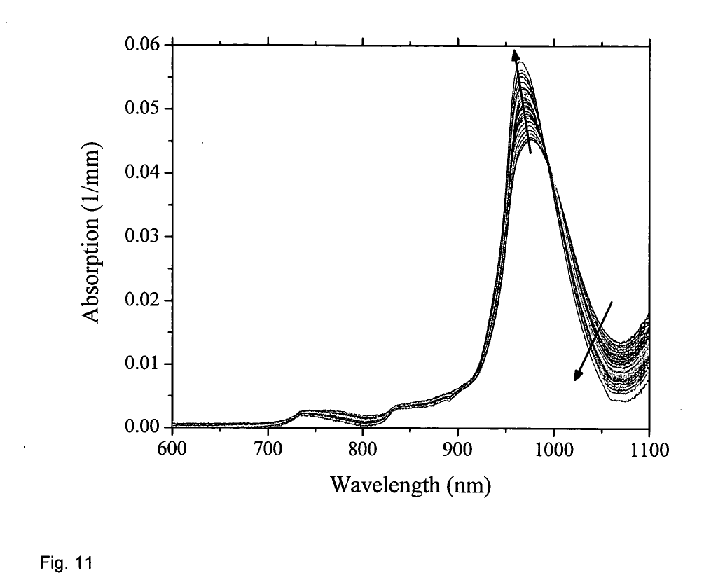

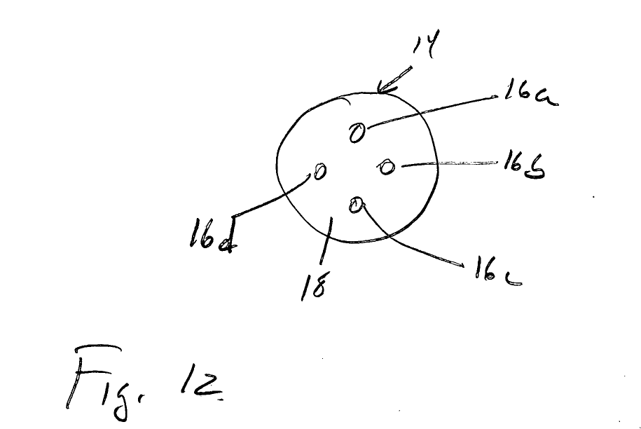

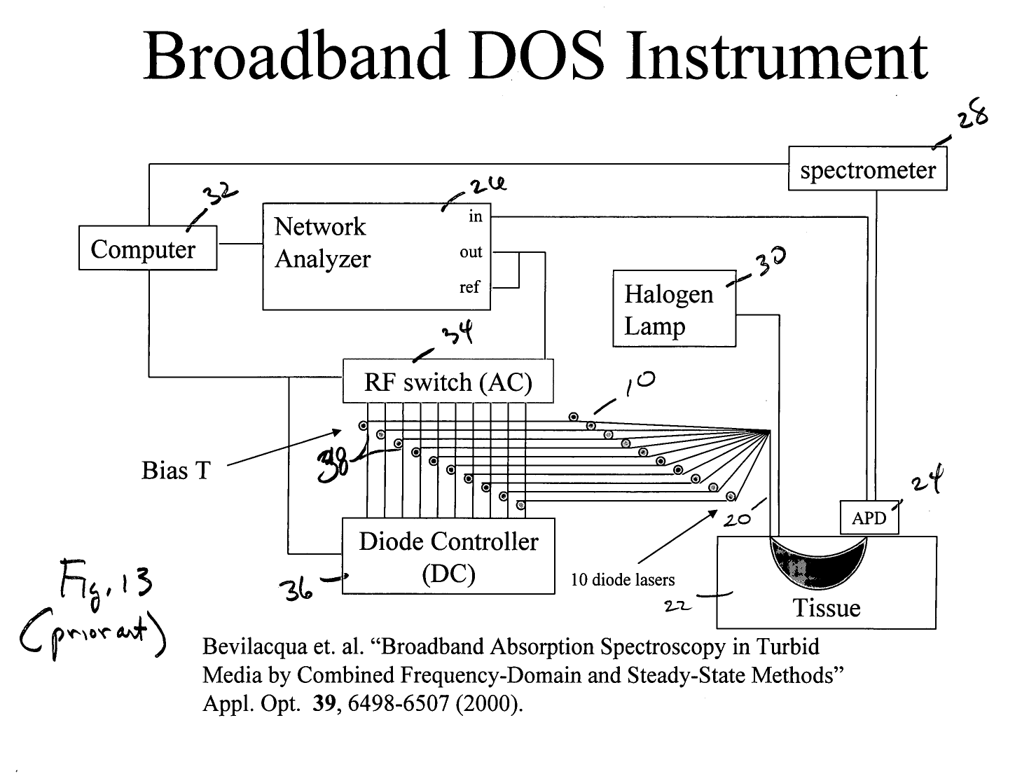

FIGS. 3a and 5b are plots of the same type as in FIGS. 1a ad 1b except for the data acquired on the animal model.

FIG. 4 is a graph of the temperature measurements acquired on hind leg of rabbit model. Temperature readings are given by thermistor #1 (▪), thermistor #2 (▴) and broadband DOS (∘). The animal was sacrificed at the point of the dashed line and ice packs were placed on the tissue to cool.

FIG. 5 is a graph of the temperature measurements acquired on hind leg of rabbit model. Temperature readings (° C.) are given by thermistor (▪) and broadband DOS (∘). The animal was sacrificed at the point of the dashed line and ice packs were placed on the tissue to cool.

FIG. 6 is a plot of temperature difference between thermistor and broadband DOS for the liquid phantom in Fig (▪) and rabbit hind-leg muscle presented in FIG. 4 (∘) and FIG. 5 (Δ).

FIG. 7 is a plot of thermistor temperature (° C.) vs. broadband DOS temperature (° C.) for all phantom and rabbit muscle measurements. The red line is a linear fit through the points and the correlation coefficient is R=0.996.

FIG. 10 is a side cross-sectional view of a plastic cuvette filled with water.

FIG. 8 is a graph of corrected temperature dependent water spectrum determined from spectrophotometer measurements.

FIG. 12 is a plan view of the end of a probe in which optic fibers used in the FD-SS measurements are contained.

FIG. 13 is a block diagram of a system by which broadband diffuse optical spectroscopy using combined FD-SS measurements are made.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

Materials and Methods

a. Broadband DOS

Broadband DOS is a technique that combines frequency domain (FD) measurements with a broadband steady-state (SS) measurement and determines the absolute absorption and scattering of the tissue within the near-infrared (600-1050 nm). From the measured tissue absorption the absolute tissue chromophores can be determined. Broadband DOS using combined frequency domain (FD) measurements with a broadband steady-state (SS) measurement has recently been developed by researchers at the Beckman Laser Institute at the University of California at Irvine, and has been published in Bevilacqua et.al., Applied Optics, Vol 39, No. 34, pp 6498-6507 (December 2000). See also Tromberg et al, “Broadband Absorption Spectroscopy In Turbid Media By Combined Frequency-Domain And Steady State Methodologies” U.S. Patent Application 20030023172 (Jan. 30, 2003) incorporated herein by reference.

As described in the published literature most of the wavelength coverage is provided by a white-light SS measurement, whereas the FD data are acquired at a few selected wavelengths. Coefficients of absorption μₐ and reduced scattering μ'ₛ derived from the FD data are used to calibrate the intensity of the SS measurements and to estimate μ'ₛ at all wavelengths in the spectral window of interest. After these steps are performed, one can determine μₐ by comparing the SS reflectance values with the predictions of diffusion theory, wavelength by wavelength. Absorption spectra of a turbid phantom and of human breast tissue in vivo, derived with the combined SSFD technique, agree well with expected reference values. All measurements can be performed at a single source-detector separation distance, reducing the variations in sampling volume that exist in multidistance methods. The technique uses relatively inexpensive light sources and detectors and is easily implemented on an existing multiwavelength FD system.

By way of background FIG. 13 is a diagrammatic block diagram which shows the experimental arrangement for Broadband DOS SSFD measurements as is conventionally know in the published literature. In all cases, light is delivered via optical fiber 16a shown on the probe head 14 in FIG. 12 to the surface of the sample 22 and collected at some distance away. In the FD mode, the light arrives sequentially from one of six amplitude-modulated diode lasers at 661, 681, 783, 803, 823 and 850 nm all with output powers of up to 20 mW at the sample 22. The light is detected by an avalanche photodiode unit 24 (Hamamatsu C556P-56045-03) coupled to optic fiber 16b that amplifies the AC component of the signal. A network analyzer 26 (Hewlett-Packard 8753C) coupled to photodiode unit 24 delivers 251 modulation frequencies from 50 to 500 MHz and measures phase and modulation amplitudes of the photon intensity signal as described in the publication cited above.

In the SS mode light comes from a 150-W halogen lamp 30 (Fiber-Lite) and is analyzed by a fiber-coupled spectrograph 28 (Ocean Optics S2000) with a linear CCD detector from 525 to 1155 nm, with the useful range for our experiments being 650-1000 nm. The spectrograph 28 records a total of 2048 points (0.35 nm/pixel), and the spectral resolution is 5 nm full width at half-maximum.

We measure the spectrum of the light source separately by inserting the source and detector fibers 16b and 16d into different ports of an integrating sphere (Labsphere, IS-040-SF). Relative reflectance is calculated to be the sample spectrum divided by the source spectrum. Total acquisition time per sample for SSFD measurements is of the order of 40 s, namely 30 s for FD and 10 s for SS. We calculate Pa according to the above published methods with an in-house Matlab (The MathWorks, Inc.) code, making use of the optimization toolbox. Computer 32 is coupled to spectrometer 28 and network analyzer 26 for data processor and to an AC controlled RF switch 34 and DC diode controller 36 for control of broadband frequency sweeping of diode lasers 10 by means of bias T's 38 coupled to RF switch 34 and diode controller 36.

For the FD measurements six diode lasers 10 at wavelengths of 661, 681, 783, 803, 823 and 850 nm were used and intensity modulated from 50 to 500 MHz. The delivery and collection FD and SS optical fibers 16a-16d were secured in a Delrin® acetal resin probe 14 diagrammatically depicted in FIG. 12. The probe head 18 was a one-inch (2.54 cm) diameter cylinder with holes drilled through lengthwise for placement of the four fibers 16a-16d. Two detection fibers 16b and 16d for the FD and SS measurement respectively were each fixed in the probe 14 at an equal distance from their corresponding source fibers 16a and 16c. The source-to-detector separation on the probe head 18 was 6 mm for liquid phantom measurement and 10 mm for the animal measurements. It must be understood that many different types and configurations of probe geometry may be employed and that the present embodiment is provided only for purposes of example. In addition, not only probe geometry can be varied but the number optical elements or fibers can be varied.

For the FD measurements, the amplitude and phase of light were calibrated either using a liquid phantom consisting of two parts Lyposyn 10% IV (LOT 05-372-DE) with one part water or a silicon phantom. The optical properties of the liquid calibration phantom were determined prior to the sample measurements through a set of multi-frequency, multi-distance, infinite geometry measurements.

The chromophore concentration fit was performed to the absolute μₐ spectrum using the extinction coefficients of the main absorbers within the tissue or sample. In this study the samples are liquid phantoms and rabbit muscle, therefore the chromophore fit extracts concentrations of water, lipid, and Nigrosin for the phantom model and Hb, HbO₂, water and lipid for the animal model.

Liquid Phantom Model

The liquid phantom measured was composed of 250 ml water, 250 ml Lyposyn 10% IV (LOT 08-412-DE) and 1.27 mg Nigrosin. The Lyposyn 10% IV emulsion was added for scattering and the Nigrosin was added to mimic tissue absorption.

The liquid phantom was placed in a Pyrex beaker on a heating and stirring plate. The broadband DOS probe 14 was placed directly on the surface of the liquid phantom and the distance between the optical source fibers 16a and 16c and detector fibers 16d and 16d was 6 mm. A thermistor probe (not shown) was placed adjacent to the optical fibers at a depth of ˜1-2 mm into the phantom, which was approximately the mean probing depth of the light. A magnetic stir bar (not shown) was placed in the phantom and used before each measurement to stir the phantom and ensure a homogeneous composition of both optical properties and temperature. Baseline measurements were acquired at room temperature before heating the phantom and the thermistor resistance was recorded at the beginning and end of each optical measurement; an average resistance was calculated from the two values and converted to temperature. The phantom was then heated with the hot plate from room temperature up to 47° C. and then allowed to cool down. Broadband DOS measurements were acquired throughout the heating and cooling process.

Animal Model

A pathogen-free white New Zealand rabbit (Myrtle Rabbitry Inc., Thompson Station, Tenn.) was used. The animal was housed in a pathogen-free animal facility and given a commercial basal diet and water ad libitum. All procedures followed a protocol approved by the Institutional Laboratory Animal Care and Use Committee, University of California, Irvine (ARC protocol No. 2000-2218).

Details of the experimental procedure involving sedation and drug injection in the animal were conventional. A plastic probe 14 incorporating the source and detector fibers was placed on the medial surface of the right hind thigh for the broadband DOS measurements. A source and detector separation of 10 mm was used for both FD and SS acquisitions. Prior to euthanizing the animal by standard procedures at the end of studies (Eutha, intravenous injection), two thermistors were imbedded in the leg of the animal adjacent to the broadband DOS probe. One thermistor was imbedded ˜3 mm deep into the muscle tissue and the other was more superficial and placed directly under the skin.

Baseline measurements were taken with the broadband DOS system prior to sacrifice and then after sacrifice, plastic bags filled with ice were placed around the DOS probe on the surface of the animal's tissue and underneath the animal's leg. Broadband DOS measurements as well as thermistor resistance values were then acquired continuously as the tissue cooled. Thermistor readings were acquired at the beginning and end of each broadband DOS measurement and averaged.

Temperature Fitting Algorithm

The algorithm used to fit the broadband DOS data for temperature was the following. Starting with the broadband absorption measured using the DOS instrument the bound water shift (bws), was calculated for the baseline measurements prior to cooling as described in the doctoral thesis by Sean Merritt on file at the library or the University of California, Irvine. For the bws fit the tissue temperature was assumed equal to the baseline temperatures measured using the thermistors. In the case of these experiments the true temperature was known from the thermistors, but in a clinical system it would be necessary to assume the temperature based on previous knowledge. With the bws, we could then correct the water chromophore used in the algorithm for effects due to macromolecule binding described the doctoral thesis of Sean Merritt.

A temperature dependent pure water spectra were measured in a spectrophotometer. Measurement of purely absorbing samples within a spectrophotometer allow for a direct measurement of the extinction coefficients. The water spectrum we have used in the past was measured at 22° C., much lower than tissue temperatures of interest (33-35° C.). We focus on the measurement of water absorption over a range of temperatures (17-60° C.) that are used for the temperature fitting in this disclosure.

The spectrophotometer used was a Beckman DU650 which has a piezo temperature controller system. In order to get an accurate measure of the water temperature, two cuvettes 20 were filled and placed size by side in the cuvette cell holder 22. One water sample was within the light path of the spectrophotometer and used for absorption measurements while the other sample had a thermistor (Newport, ±0.2° C.) immersed in the water sample at the same level as the light path on the measured sample. Resistance was recorded from the thermistor during the spectrophotometer measurements and converted to temperature which was used as the true temperature of the measurement sample.

Ideally, calibration of the measurement would be performed using different cell path lengths, which would allow for the absorption of the plastic cuvette to be removed. Because the cuvette cell 22 on the spectrophotometer available was a fixed distance a blank was acquired using an empty cuvette 20 in order to account for the absorption of the cuvette 20 itself as well as the source intensity.

FIG. 10 illustrates the water filled cuvette 20 with the index of refractions for air, plastic and water given as nₐᵢᵣ, nₚ and n_{w}. The source intensity is given by I₀ and enters the cuvette 20 at the left side of FIG. 10. The output intensity on the right side is given by I where:
I=T₁×T₂×T₃×T₄×T_{w}×Tₚ×I₀, (0<Tᵢ<1)

Where T₁ through T₄ are transmission factors determined from the perpendicular Fresnel reflections occurring at the boundaries and are given by:

$${T}_{⊥} = \frac{4 {n}_{1}^{2}}{{( {n}_{1} + {n}_{2} )}^{2}}$$

with n₁ and n₂ being the index of refraction of the medium the light is exiting and entering respectively. T_{w} and Tₚ account for the transmitted light through the water and the plastic due to the absorption of these materials.

As stated above the calibration measurement was performed on an empty cuvette. The only difference between the measurement sample and the blank is then that the water index of refraction is replaced with that of air. The transmitted intensities for a pure water sample and an empty cuvette are given by Iₛ and I_{b} respectively:

$${I}_{s} = \frac{256 {n}_{p}^{4} {n}_{w}^{2}}{{( {n}_{p} + 1 )}^{4} {( {n}_{p} + {n}_{w} )}^{4}} \times {T}_{w} \times {T}_{p} \times {I}_{0}$$

$${I}_{b} = \frac{256 {n}_{p}^{4}}{{( {n}_{p} + 1 )}^{8}} \times {T}_{p} \times {I}_{0}$$

The measured parameter reported by the spectrophotometer is the absorbance (A_{spec}) which is given by taking the ratio of the measured sample intensity with the intensity measured on the blank cuvette:

$$\frac{{I}_{s}}{{I}_{b}} = \frac{{{n}_{w}^{2} ( {n}_{p} + 1 )}^{4}}{{( {n}_{p} + {n}_{w} )}^{4}} \times {T}_{w} = {10}^{- {A}_{spec}}$$

Solving for A_{spec} gives:

$${A}_{spec} = - log ( \frac{{{n}_{w}^{2} ( {n}_{p} + 1 )}^{4}}{{( {n}_{p} + {n}_{w} )}^{4}} ) - log ( {T}_{w} )$$

If the sample and the blank measurement had the same index of refraction, as is normally the case, the first log term would be equal to zero and the absorbance would be equal to −log(T_{w}), which is the desired parameter being measured. Therefore, the true absorbance of pure water can be determined given knowledge of the index of refraction of water and the material of the cuvette, which is polymethyl methacrylate (PMMA) using the following:

$${A}_{water} = - log ( {T}_{w} ) = - log ( \frac{{{n}_{w}^{2} ( {n}_{p} + 1 )}^{4}}{{( {n}_{p} + {n}_{w} )}^{4}} ) - {A}_{spec}$$

The indexes of refraction of water and PMMA have been well characterized within the wavelength region of interest.

Plugging n_{w} and nₚ from the above plots into the equation above results in

$${A}_{water} = - log ( {T}_{w} ) = - log ( \frac{{{n}_{w}^{2} ( {n}_{p} + 1 )}^{4}}{{( {n}_{p} + {n}_{w} )}^{4}} ) - {A}_{spec}$$

which provides an absolute pure water spectrum. The corrected water spectra that were measured in the spectrophotometer are presented in FIG. 8. The direction of the arrows in the figure indicate the direction of the changing absorption as the temperature is increased. As the temperature is increased from 17 to 60° C. there is a blue shift and narrowing of the absorption peak as well as an increase in intensity. There is also an isosbestic point at about 1000 nm, which is consistent with references and suggests the majority of the water hydrogen is in one of two states: unbound or bound.

In order to test the accuracy of the above correction, the 22° C. water spectrum measured in the spectrophotometer can be interpolated from the temperature dependent data in FIG. 8 and compared with the water spectrum measured by known published standards. The primary wavelength range of interest is between 935-1000 nm given that this is the spectral window used for the temperature fitting. Over this wavelength range, the percentage difference is within ±5% of the water absorption measured by published standards. The largest percentage difference between the two curves is around 600 nm where the absorption is very small and the differences can be as large as 40%.

The measured water spectra in FIG. 11 were first corrected for the bound water shift of the tissue and then used as the temperature dependent water chromophore. Water spectra at specific temperatures were interpolated from this library of temperature dependent water spectra to provide water absorption spectra at all temperatures between 17 and 60° C.

The broadband DOS data was weighted such that only the absorption values around the water absorption peak were used because weighting the full spectra equally converged on temperatures which correlated with the true temperatures, but were underestimated. For both the liquid phantom and animal model a spectral window defined from 935-1000 nm was used in the fitting. With the spectral window defined, a Matlab least squares fitting routine (‘Isqcurvefit’) determines the temperature of the sample by finding the best fit between the measured water absorption spectrum and the addition of a flat baseline chromophore and a water absorption spectrum at a specific temperature from the temperature dependent water chromophore set. The details behind defining the spectral weighting window are described in more detail below.

The fitting algorithm continues by performing a chromophore fit in which the water absorption spectrum used is at the temperature determined by the previous temperature fit. After new chromophore values are determined, the temperature fit is repeated with the new chromophore values fixed and the algorithm is set in a loop that fits for chromophore values and follows with a temperature fit. The final temperature is determined when the difference between two consecutive fit temperatures is less than a fixed value which was set at 0.1° C. for the measurements presented in embodiment.

This temperature fitting algorithm was insensitive to the initial guess of the chromophores and temperature and in all the measurements presented, the temperature fitting algorithm converged to a temperature within five iterations in the loop described.

Results

a. Phantom Model

In order to determine the optimal spectral window for the weighting of the liquid phantom data, the data was processed by fixing the upper or lower bound of the spectral window at 1000 nm or 945 nm respectively. The opposite window position was then incrementally increased or decreased from a starting wavelength of 975 nm. For each spectral window, temperatures were recovered using broadband DOS as described above. These recovered temperatures were then correlated and compared with the thermistor temperatures. FIG. 1a is a plot of the correlation coefficient (R) from the broadband DOS vs. thermistor temperatures where the spectral window upper bound is fixed at 1000 nm and the lower bound of the window is varied from 975 to 935 nm. FIG. 1b is a plot of the mean temperature difference (thermistor—DOS) between the two measurements and the standard deviation of the temperature differences over the same spectral window range. As the lower bound of the spectral window is increased the correlation of the temperatures increases and plateaus and the mean differences between the temperatures decrease and levels off.

FIGS. a and 2b are the same type of plots as shown in FIGS. 1a and 1b respectively except the lower bound of the spectral window is fixed at 945 nm and the upper bound is increased from 975 nm to 1015 nm. Increasing the upper bound of the spectral window has similar effects to those in FIGS. 1a and 1b, namely the correlation increases and the mean error decreases.

The above analysis of the weighted data led to the use of a spectral window between 935 and 1000 nm to be used for all the data presented in this chapter. FIGS. 1a and 1b illustrate that the lower bound wavelength of the spectral window should be below 950 nm and FIGS. 2a and 2b show that the upper bound wavelength of the spectral window should be above 985 nm. Expanding the window to 935 and 1000 nm provided an overlap of the spectral region in which the bound water correction describe in Merritt et al was implemented.

FIG. 3 is a plot of the temperature (° C.) vs. time of measurement (minutes) from data acquired on a liquid phantom. The black squares are the temperatures determined from the thermistor resistance and the error bars on the measurements are within the squares. The circles are the temperatures derived using broadband DOS. The bws determined from the first measurement and applied in the fitting for all following measurements was 0.5 nm. For the case of the liquid phantom the molecular water binding is due to the water interacting with the emulsifying agent within the 10% Lyposin solution. Taking the difference between the recovered broadband DOS temperature and the measured thermistor temperature at all the points results in an average absolute difference of 2.0±1.4° C. Although the correlation is high (R=0.996) between the thermistor temperature and the broadband DOS temperature, there is a relatively large difference between the values, nearly 5° C. at the peak temperature.

The differences between the thermistor (▪) and the broadband DOS (∘) temperatures in the liquid phantom measured in Fig are linearly increasing with temperature. In order to investigate this effect further two additional liquid phantoms were measured on separate days over different temperature ranges. The temperature difference between the thermistor and broadband DOS values are plotted for all three liquid phantoms in FIG. 2. For all phantoms, as the temperature of the phantom increases the difference between the thermistor temperature and the broadband DOS temperatures increase at the same rate. The temperature at which the difference is minimized is around 30° C. for these phantoms. All phantoms had a 0.5 nm bound water shift correction implemented, but if this shift is increased to 1.5 nm the entire temperature difference curve is displaced downward, such that the temperature at which the difference is minimized becomes ˜50° C. Therefore, the bound water shift correction only moves the curve in FIG. 2 in the vertical direction, but does not change the slope.

b. Animal Model

The liquid phantom data above provides an ideal case where the sample is homogeneous in composition and temperature as well as being mostly water, which is the source of the contrast in temperature. In order to provide a proof of principle for this technique to be used in vivo we now present data acquired on the animal model described above.

As with the liquid phantom the spectral window used in the temperature fitting portion of the algorithm was examined. FIGS. 5a and 5b are the same type of plots as FIGS. 1a and 1b with data from the rabbit model substituted for the liquid phantom data. Similar to the liquid phantom data there is a plateau that is reached for the correlation between the DOS temperatures and the thermistor temperatures. Also, the mean difference between the temperatures levels off and the standard deviation of the differences is relatively flat. The graphs in which the upper bound wavelength of the spectral window is increased are also similar to the data acquired on the liquid phantom. Therefore, the same spectral window from 935-1000 nm was used for the analysis on the animal model data.

FIG. 6 is a plot of the temperature (° C.) vs. time of measurement for the data acquired on the animal model described above. Here there are three values plotted: the broadband DOS measurement (∘), thermistor #1 (▪) placed ˜3 mm deep into the tissue and thermistor #2 (▴) superficially placed directly under the skin. Four baseline measurements were taken prior to sacrifice, which occurred at the point of the vertical dashed line on the plot. After the fifth measurement was taken ice packs were applied to the tissue surface and underneath the animal leg to cool down the tissue.

Broadband DOS measurements tracked well with thermistor #2 measurements prior to the application of the ice packs and then track better with thermistor #1 temperatures. Following the placement of the ice packs, the registered temperature begins to drop in both of the thermistor readings and the broadband DOS measurement. Thermistor #2, which was placed directly under the skin drops 10° C. directly after the ice packs were applied to the tissue. Thermistor #1 and the broadband DOS readings both begin to drop in temperature at similar rates with a final temperature of 25° C. being reached after the ice packs had been applied for 40 minutes.

The mean bws determined from the first four measurements and applied in the fitting for all following measurements was 1.5±0.1 nm. Taking the difference between the recovered broadband DOS temperature and the measured thermistor #1 temperature at all the points results in an average absolute difference of 0.7±0.7° C. and the correlation between the temperatures was R=0.989. The thermistor reading in this case can only act as a guide for comparison with the broadband DOS measurements, but is not actually the true tissue temperature over the entire broadband DOS light field since the temperature in the animal model is more heterogeneously distributed.

A second rabbit was measured for further validation (see FIG. 7). In this case the average bws measured for the five baseline measurements was 2.7±0.1 nm. The same spectral window from 935-1000 nm was used for this rabbit for consistency. The difference between the recovered broadband DOS temperature and the thermistor temperature of the 32 time points measured gave an average absolute difference of 0.4±0.3° C. As seen in FIG. 5 there is gap of data points missing directly following the sacrifice of the animal. During this time the thermistor, which was imbedded in the muscle came loose and a repositioning of the DOS probe and thermistor was necessary. Therefore, the measurements following death are probing a slightly different tissue volume, although the measurements were taken at approximately the same location. The correlation of the thermistor temperature and the broadband DOS temperature for the second rabbit measurement was R=0.995.

The temperature differences between the thermistor and broadband DOS measurements for the animal model data are similar to the trends in the liquid phantom data. FIG. 6 is a plot of the temperature differences in the liquid phantom presented in Fig (▪) and rabbit hind-leg muscle data presented in FIG. 4 (∘) and FIG. 5 (Δ). The data from the animal models is distributed along the curve that was initially observed for the phantom data.

Given that the temperature difference between all the samples changes in a systematic way following the bound water correction, a linear fit can be performed between the thermistor temperatures and the broadband DOS temperatures to determine a linear correction for the broadband DOS data. FIG. 7 is a plot of the thermistor temperature vs. the broadband DOS temperatures for all the measurements presented. The best linear fit to the data is given by the black line which is defined by the following equation:

Thermistor Temperature=1.32±0.01×Broadband DOS Temperature−8.99±0.36

The correlation coefficient from the fit is R=0.996. Applying this correction to the broadband DOS temperatures gave an absolute difference between the measured temperature and corrected temperature of 0.68±0.58° C. and 0.66±0.56° C. for the liquid phantom and animal model temperatures respectively.

The above disclosure describes a method for determining bulk deep tissue temperatures. From the motivation presented in the introduction and the preliminary in vivo results presented, this method is clearly useful for application to skin remodeling which could benefit from the measurement of skin as well as subcutaneous tissue temperatures. With the current algorithm an accuracy of 0.7±0.6° C. for rabbit skin/muscle was achieved. Through a better understanding of a water molecules interactions in a particular tissue type as well as larger numbers of measurements that could possibly be used as a calibration of the instrument, the accuracy in predicted temperature would be even better.

The method is also applicable to any tissue including particularly to human breast tissue. Compared to the phantoms and tissue investigated in this disclosure, a normal breast would be more challenging because of its lack of water. Breast tissue has water content as low as 10% by volume and therefore, the contrast of tissue temperature will be significantly reduced. The larger tissue volumes will also require tomographic capabilities in order to map out normal and tumor tissue temperatures. This will require a tomography system with broad spectral capabilities, which is entirely within the spirit and scope of the present invention.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

For example, vulnerable plaques are typically small lesions comprised of a lipid-rich core, surrounded by a thin, collagenous cap with varying degrees of smooth muscle cells. The vulnerable plaques form within the walls of cardiovascular vessels. The plaques may be characterized by a thickened arterial wall, partial stenosis, and generally elliptical distortion of the cardiovascular lumen with blockages ranging from zero up to about 70%. Stenoses are generally less severe with vulnerable plaques than stable plaques. However, mild stenoses are far more common and are responsible for more occlusions than tighter stenoses. Vulnerable plaques may be differentiated by their size, shape and composition of their lipid cores and fibrous caps. Acute lesions are larger with crescent-shaped cross-sections rich in extracellular lipid accumulation. The fibrous cap may be infiltrated with macrophages throughout, a precursor to initiating the disruption of the vulnerable plaque with mechanical strain or degradation of the wall thickness.

The fibrous cap may fatigue and rupture from mechanical stresses, releasing macrophages and tissue factor leading to thrombosis. The cap may be compromised by the presence of inflammation and swelling. As a result, activated inflammatory cells release heat that, when detected, indicates the presence and progression of vulnerable plaque.

The illustrated embodiment of the invention thus provides a method to measure intravascular temperature noninvasively. Broadband Diffuse Optical Spectroscopy (DOS) is a non-invasive method used for quantifying absorption and scattering parameters of tissue over a broad wavelength range (600-2500 nm). Using broadband DOS, endovascularly delivered via a catheter to a region of interest, to give an absolute measure of the tissues absorption, it is possible to determine the concentrations of the chromophores in the tissue such as oxy- and doxy-hemoglobin, water, fat in addition to reduced scattering coefficient which is related to the tissue matrix. The absorption profiles of these chromophores are temperature dependent and can therefore be used as temperature meters.

Fundamentally, what we are measuring is the vibrational energies of bonds within these chromophores and as tissue temperature changes these energies will change due to complex interactions of the chromophores as bonds are created and broken with other molecules. By characterizing how these tissue chromophores change with temperature we can then use broadband DOS to measure tissue temperature.

The invention can also be extended to encompass tomographic methods whereby two or three dimensional images of tissue may be generated using an array of scanning elements, each element measuring the absolute absorption of light at each pixel in the image according to conventional tomographic processing.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. A method for noninvasively determining tissue temperature comprising:
measuring data relating to spectral shifts of chromophore absorption in tissue using broadband diffuse optical spectroscopy; and
generating a temperature reading corresponding to the spectral shift of an absorption peak of the chromophore.

2. The method of claim 1 further comprising performing a bound water correction to the spectral shift.

3. The method of claim 1 where measuring spectral shifts comprises making a frequency domain measurement at multiple wavelengths to determine the absolute absorption and scattering values at the multiple wavelengths.

4. The method of claim 3 where measuring spectral shifts further comprises scaling with the frequency domain measurements to provide an absolute quantitative absorption spectrum in the wavelength region between 600 and 1050 nm.

5. The method of claim 1 where measuring spectral shifts comprises making a measurement of an absolute scattering and absorption.

6. The method of claim 5 where making a measurement of an absolute absorption comprises measuring an absolute absorption coefficient of selected tissue and further comprising deducing concentrations of tissue composition including lipids.

7. The method of claim 5 where making a measurement of an absolute scattering comprises measuring an absolute scattering coefficient of selected tissue and further comprising deducing information related to heterogeneity and integrity of tissue matrix.

8. The method of claim 1 further comprising deducing temperature heterogeneity related to vulnerable plaque in vascular tissue.

9. The method of claim 5 where making a measurement of an absolute scattering and absorption comprises making a measurement in the range of 600-1100 nm to interrogate a vessel wall in the presence of blood.

10. The method of claim 1 where generating a temperature reading corresponding to the spectral shift of an absorption peak of the chromophore comprises:
providing bound water shift, bws, for water chromophore as a baseline measurements corrected for effects due to macromolecule binding to provide a library of temperature dependent water absorption spectra at all temperatures over a predetermined temperature range;
weighting the broadband DOS data such that only the absorption values within a predetermined spectral window which included a selected water absorption peak were used; and
finding the best fit between (1) the measured water absorption spectrum and (2) the sum of a flat baseline chromophore and a water absorption spectrum at a specific temperature from library of temperature dependent water absorption spectra.

11. The method of claim 10 further comprising performing a chromophore fit in which the water absorption spectrum used is at the temperature determined by the previous temperature fit, determining new chromophore values, and repeating finding the best fit with the new chromophore values being fixed, where finding the best fit is performed in a loop which fits for chromophore values and follows fits for temperature, a final temperature being determined when the difference between two consecutive fit temperatures is less than a predetermined value.

12. The method of claim 11 where predetermined value for the difference between two consecutive fit temperatures is 0.1° C.

13. The method of claim 10 where weighting the broadband DOS data such that only the absorption values within a predetermined spectral window comprises weighting the broadband DOS data such that only the absorption values within a spectral window defined from 935-1000 nm.

14. A method for noninvasively determining an unknown tissue chromophore comprising:
measuring data relating to spectral shifts of the unknown chromophore absorption in tissue using broadband diffuse optical spectroscopy;
generating a temperature reading corresponding to a spectral shift of an absorption peak of the unknown chromophore; and
matching a spectral shift of an absorption peak of a known chromophore selected from a library to the spectral shift of an absorption peak of the unknown chromophore to obtain a best fit among a plurality of known chromophore candidates to identify the unknown chromophore with the known chromophore.

15. A method for noninvasively detecting vulnerable plaque in vascular tissue comprising:
measuring data relating to spectral shifts of chromophore absorption in tissue using broadband diffuse optical spectroscopy corresponding to at least one chromophore correlated with vulnerable plaque in vascular tissue; and
generating at least one temperature reading corresponding to the spectral shift of an absorption peak of the at least one chromophore to identify temperature heterogeneity in the vascular tissue and presence of vulnerable plaque.

16. An apparatus for noninvasively determining deep tissue temperature comprising:
means for measuring data relating to spectral shifts of chromophore absorption in tissue using broadband diffuse optical spectroscopy; and
means for generating a temperature reading corresponding to the spectral shift of an absorption peak of the chromophore.

17. The apparatus of claim 16 further comprising means for performing a bound water correction to the spectral shift.

18. The apparatus of claim 16 where the means for measuring spectral shifts comprises means for making a frequency domain measurement at multiple wavelengths to determine the absolute absorption and scattering values at the multiple wavelengths.

19. The apparatus of claim 18 where the means for measuring spectral shifts further comprises means for scaling with the frequency domain measurements to provide an absolute quantitative absorption spectrum in the wavelength region between 600 and 1050 nm.

20. The apparatus of claim 16 where the means for measuring spectral shifts comprises means for making a measurement of an absolute scattering and absorption.

21. The apparatus of claim 20 where the means for making a measurement of an absolute absorption comprises means for measuring an absolute absorption coefficient of selected tissue and further comprising means for deducing concentrations of tissue composition including lipids.

22. The apparatus of claim 20 where the means for making a measurement of an absolute scattering comprises means for measuring an absolute scattering coefficient of selected tissue and further comprising means for deducing information related to heterogeneity and integrity of tissue matrix.

23. The apparatus of claim 16 further comprising means for deduce temperature heterogeneity related to vulnerable plaque in vascular tissue.

24. The apparatus of claim 20 where the means for making a measurement of an absolute scattering and absorption comprises means for making a measurement in the range of 600-1100 nm to interrogate a vessel wall in the presence of blood.

25. The apparatus of claim 16 where the means for generating a temperature reading corresponding to the spectral shift of an absorption peak of the chromophore comprises:
means for providing bound water shift, bws, for water chromophore as a baseline measurements corrected for effects due to macromolecule binding to provide a library of temperature dependent water absorption spectra at all temperatures over a predetermined temperature range;
means for weighting the broadband DOS data such that only the absorption values within a predetermined spectral window which included a selected water absorption peak were used; and
means for finding the best fit between (1) the measured water absorption spectrum and (2) the sum of a flat baseline chromophore and a water absorption spectrum at a specific temperature from library of temperature dependent water absorption spectra.

26. The apparatus of claim 25 further means for comprising performing a chromophore fit in which the water absorption spectrum used is at the temperature determined by the previous temperature fit, means for determining new chromophore values, and means for repeating finding the best fit with the new chromophore values being fixed, where the means for finding the best fit performs a loop which fits for chromophore values and which then fits for temperature, a final temperature being determined when the difference between two consecutive fit temperatures is less than a predetermined value.

27. The apparatus of claim 25 where the means for weighting the broadband DOS data such that only the absorption values within a predetermined spectral window comprises means for weighting the broadband DOS data such that only the absorption values within a spectral window defined from 935-1000 nm.

28. An apparatus for noninvasively determining an unknown tissue chromophore comprising:
means for measuring data relating to spectral shifts of the unknown chromophore absorption in tissue using broadband diffuse optical spectroscopy;
means for generating a temperature reading corresponding to a spectral shift of an absorption peak of the unknown chromophore; and
means for matching a spectral shift of an absorption peak of a known chromophore selected from a library to the spectral shift of an absorption peak of the unknown chromophore to obtain a best fit among a plurality of known chromophore candidates to identify the unknown chromophore with the known chromophore.

29. An apparatus for noninvasively detecting vulnerable plaque in vascular tissue comprising:
means for measuring data relating to spectral shifts of chromophore absorption in tissue using broadband diffuse optical spectroscopy corresponding to at least one chromophore correlated with vulnerable plaque in vascular tissue; and
means for generating at least one temperature reading corresponding to the spectral shift of an absorption peak of the at least one chromophore to identify temperature heterogeneity in the vascular tissue and presence of vulnerable plaque.
